(19) United States
(12) Patent Application Publication
(10) Pub. No.: US 2002/0147337 A1
(43) Pub. Date: Oct. 10, 2002

(54) Process for preparing sulfonylcarboxamide derivatives

(76) Inventors: Theodor Andreas Wollmann, Hattersheim (DE); Regina Duffy, Hattersheim (DE); Claudia Falkenstein, Niederselters (DE); Johannes Keil, Frankfurt (DE)

Correspondence Address: Finnegan, Henderson, Farabow,, Garrett & Dunner, L.L.P., 1300 I Street, N.W., Washington, DC, 20005-3315, US

(21) Appl. No.: 10/096,307

(22) Filed: Mar. 13, 2002

(30) Foreign Application Priority Data
Mar. 14, 2001 (DE) 101 12 040.0

(51) Int. Cl. C07D 265/30; C07D 217/22; C07D 215/36; C07D 241/18; C07D 211/54; C 07D 2 7/24; C07C 311/16

(52) U.S. Cl. 544/158; 544/383; 546/141; 546/153; 546/242; 548/541; 564/91

(57) ABSTRACT

An improved process for preparing sulfonylcarboxamide derivatives of the formula I

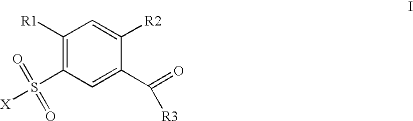

in which the radicals have the given meanings. The compounds are suitable, for example, for use as antilipemics.

This patent application claims the benefit of priority under 35 U.S.C. §119(a) to German patent application no. 10112040.0-43, filed on Mar. 14, 2001, the contents of which are incorporated by reference herein.

The invention relates to an improved process for preparing sulfonylcarboxamide derivatives and their intermediates.

Sulfonylcarboxamide derivatives of the formula I

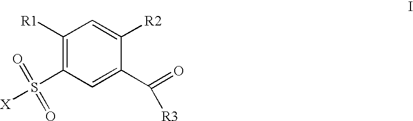

in which

X, R1, R2, R3 independently of one another are NR6R7, (CH₂)-pyridyl, (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C₃-C₆)cycloalkyl, CON H₂, CONH(C₁-C₆)alkyl, CON[(C₁-C₆)alkyl]₂;

(C₁-C₈)-alkyl, pyrrolidine, piperidine, piperazine, piperazin-2-one, morpholine, tetrahydropyridine, tetrahydroquinoline, tetrahydroisoquinoline, where the rings may in each case be substituted by phenyl, (C₁-C₆)-alkyl-phenyl, —OH, (C₁-C₈)-alkyl, (C₁-C₆)-alkyl-OH, O-phenyl, S-phenyl, (CO)—(C₁-C₆)-alkyl, (CO)-phenyl, where the phenyl substituent is unsubstituted or substituted up to two times by F, Cl, Br, OH, CF₃, CN, OCF₃, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl;

R6 and R7 independently of one another are H, (C₁-C₆)-alkyl, (C₁-C₆)-alkyl-OH, (C₁-C₆)-alkyl-NH2, (C₁-C₆)-alkyl-O—(C₁-C₆)-alkyl, O—(C₁-C₆)-alkyl, (C₃-C₆)-cycloalkyl, CO—(C₁-C₆)-alkyl, (C₁-C₆)-alkyl-NH—C(O)—(C₁-C₆)-alkyl, (C₁-C₆)-alkyl-NH—(C₁-C₆)-alkyl, (C₁-C₆)-alkyl-N—[(C₁-C₆)-alkyl]₂, (C₁-C₆)-alkyl-diphenyl, (C₁-C₆)-alkyl-O-phenyl, CHO, CO-phenyl,

(CH₂)ₙ—Ar, where n=0-6 and Ar can be phenyl, biphenyl, 1- or 2-naphthyl, 1- or 2-tetrahydrofuranyl, 2-, 3- or 4-pyridyl, 2- or 3-thienyl, 2- or 3-furyl, 2-, 4- or 5-thiazolyl, 2-, 4- or 5-oxazolyl, 1-pyrazolyl, 3-, 4- or 5-isoxazolyl, (C₃-C₆)-cycloalkyl, piperidinyl, pyrrolidinyl, oxopyridinyl, 2- or 3-pyrrolyl, 2- or 3-pyridazinyl, 2-, 4- or 5-pyrimidinyl, 2-pyrazinyl, 2-(1,3,5-triazinyl), 2-, 3- or 4-morpholinyl, 2- or 5-benzimidazolyl, 2-benzothiazolyl, 1,2,4-triazol-3-yl, 1,2,4-triazol-5-yl, tetrazol-5-yl, indol-3-yl, indol-5-yl or N-methyl-imidazol-2-, -4- or -5-yl and Ar may be substituted up to two times by F, Cl, Br, OH, CF₃, N0₂, CN, OCF₃, O—CH₂—O, O—(C₁-C₆)-alkyl, S-(C₁-C₆)-alkyl, SO-(C₁-C₆)-alkyl, S0₂-(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH-CO-(C₁-C₆)-alkyl, NH-CO-phenyl, pyrrolidin-1-yl, morpholin-1-yl, piperidin-1-yl, piperazin-1-yl, 4-methyl-piperazin-1-yl, (CH₂)n-phenyl, O-(CH₂)n-phenyl, S-(CH₂)ₙ—phenyl, SO₂-(CH₂)n-phenyl, where n=0-3;

and a process for their preparation are described in DE 19941540.

The compounds described are suitable for use as medicaments for treating hyperlipidemia and arteriosclerotic disorders, as discussed in copending U.S. application Ser. No. 09/654,841, filed on Sep. 1, 2000, the contents of which are incorporated by reference herein.

It was one object of the present invention to provide an improved process having a more simple and/or shorter synthesis route. The use of less toxic reagents and/or solvents was also desirable.

Accordingly, the invention includes a process for preparing the compounds of the formula I, which comprises preparing the compounds of the formula I according to the reaction scheme below:

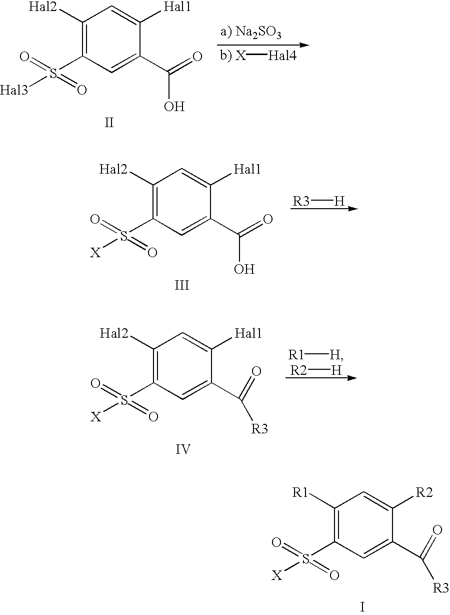

1) in the first process step, a compound of the formula II, in which Hal1 and Hal2 are each a halogen atom, preferably fluorine or chlorine, and Hal3 is a halogen atom, preferably chlorine, is reduced using sodium sulfite and then, at a pH of from 1 to 3 (preferably from 1.5 to 2.5) reacted with a compound X-Hal4, where X has the meaning given for formula I and Hal4 is a halogen atom (iodine, bromine, chlorine), preferably bromine or chlorine, in a suitable solvent (such as, for example, water, methanol, ethanol, propanol, butanol, dimethyl sulfoxide, dimethyl formamide, N-methylpyrrolidone and mixtures thereof) to give a compound of the formula III (at from 0 to 80° C., preferably from 20 to 50° C.).

2) in the second process step, the compound of the formula III obtained in process step 1) is reacted with a compound R3-H, in which R3 has the meaning given for formula 1, to give a compound of the formula IV.

3) subsequently, the compound of the formula IV obtained in process step 2) is reacted with compounds R1—H and R2—H, where R1 and R2 have the meaning given for formula 1, to give a compound of the formula 1.

To prepare compounds of the structure III where X=CH₃, it is also possible to use chloroacetic acid as alkylating agent in process step 1). The compounds initially obtained, of the structure III where X=CH₂CO₂H, are decarboxylated by heating (such as, for example, in water at 80-100° C.) and thus converted into the desired compounds where X=CH₃.

The novel process has the advantage that the three-step process described in the above application is replaced by a one-step process. If X=CH₃, highly toxic and carcinogenic reagents (CH₃Hal) can be replaced by chloroacetic acid.

The invention furthermore includes a process according to process step 1) described above for preparing intermediates of the formula IV,

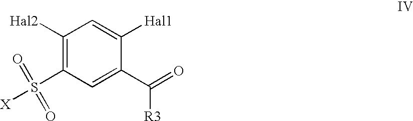

in which

X, R3 independently of one another are NR6R7, (CH₂)-pyridyl, (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C₃-C₆)cycloalkyl, CONH₂, CONH(C₁-C₆)alkyl, CON[(C₁-C₆)alkyl]₂;

(C₁-C₈)-alkyl, pyrrolidine, piperidine, piperazine, piperazin-2-one, morpholine, tetrahydropyridine, tetrahydroquinoline, tetrahydroisoquinoline, where the rings may in each case be substituted by phenyl, (C₁-C₆)-alkyl-phenyl, —OH, (C₁-C₈)-alkyl, (C₁-C₆)-alkyl-OH, O-phenyl, S-phenyl, (CO)—(C₁-C₆)-alkyl, (CO)-phenyl, where the phenyl substituent is unsubstituted or substituted up to two times by F, Cl, Br, OH, CF₃, CN, OCF₃, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl;

R6 and R7 independently of one another are H, (C₁-C₆)-alkyl, (C₁-C₆)-alkyl-OH, (C₁-C₆)-alkyl-NH2, (C₁-C₆)-alkyl-O—(C₁-C₆)-alkyl, O—(C₁-C₆)-alkyl, (C₃-C₆)-cycloalkyl, CO—(C₁-C₆)-alkyl, (C₁-C₆)-alkyl-NH—C(O)—(C₁-C₆)-alkyl, (C₁-C₆)-alkyl-NH-(C₁-C₆)-alkyl, (C₁-C₆)-alkyl-N-[(C₁-C₆)-alkyl]₂, (C₁-C₆)-alkyl-diphenyl, (C₁-C₆)-alkyl-O-phenyl, CHO, CO-phenyl,

(CH₂)ₙ—Ar, where n=0-6 and Ar can be phenyl, biphenyl, 1- or 2-naphthyl, 1- or 2-tetrahydrofuranyl, 2-, 3- or 4-pyridyl, 2- or 3-thienyl, 2- or 3-furyl, 2-, 4- or 5-thiazolyl, 2-, 4- or 5-oxazolyl, 1-pyrazolyl, 3-, 4- or 5-isoxazolyl, (C₃-C₆)-cycloalkyl, piperidinyl, pyrrolidinyl, oxopyridinyl, 2- or 3-pyrrolyl, 2- or 3-pyridazinyl, 2-, 4- or 5-pyrimidinyl, 2-pyrazinyl, 2-(1,3,5-triazinyl), 2-, 3- or 4-morpholinyl, 2- or 5-benzimidazolyl, 2-benzothiazolyl, 1,2,4-triazol-3-yl, 1,2,4-triazol-5-yl, tetrazol-5-yl, indol-3-yl, indol-5-yl or N-methyl-imidazol-2-, -4- or -5-yl and Ar may be substituted up to two times by F, Cl, Br, OH, CF₃, NO₂, CN, OCF₃, O—CH₂—O, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl, pyrrolidin-1-yl, morpholin-1-yl, piperidin-1-yl, piperazin-1-yl, 4-methyl-piperazin-1-yl, (CH₂)ₙ-phenyl, O—(CH₂)ₙ-phenyl, S—(CH₂)ₙ-phenyl, SO₂—(CH₂)ₙ-phenyl, where n=0-3;

Hal1, Hal2 independently of one another are F, Cl, Br.

The invention furthermore includes intermediates of the formula IV,

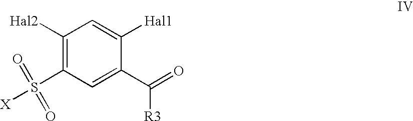

in which

X, R3 independently of one another are NR6R7, (CH₂)-pyridyl, (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C₃-C₆)cycloalkyl, CONH₂, CONH(C₁-C₆)alkyl, CON[(C₁-C₆)alkyl]₂;

(C₁-C₈)-alkyl, pyrrolidine, piperidine, piperazine, piperazin-2-one, morpholine, tetrahydropyridine, tetrahydroquinoline, tetrahydroisoquinoline, where the rings may in each case be substituted by phenyl, (C₁-C₆)-alkyl-phenyl, —OH, (C₁-C₈)-alkyl, (C₁-C₆)-alkyl-OH, O-phenyl, S-phenyl, (CO)—(C₁-C₆)-alkyl, (CO)-phenyl, where the phenyl substituent is unsubstituted or substituted up to two times by F, Cl, Br, OH, CF₃, CN, OCF₃, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl;

R6 and R7 independently of one another are H, (C₁-C₆)-alkyl, (C₁-C₆)-alkyl-OH, (C₁-C₆)-alkyl-NH2, (C₁-C₆)-alkyl-O-(C₁-C₆)-alkyl, O—(C₁-C₆)-alkyl, (C₃-C₆)-cycloalkyl, CO—(C₁-C₆)-alkyl, (C₁-C₆)-alkyl-NH—C(O)—(C₁-C₆)-alkyl, (C₁-C₆)-alkyl-NH—(C₁-C₆)-alkyl, (C₁-C₆)-alkyl-N-[(C₁-C₆)-alkyl]₂, (C₁-C₆)-alkyl-diphenyl, (C₁-C₆)-alkyl-O-phenyl, CHO, CO-phenyl,

(CH₂)ₙ—Ar, where n=0-6 and Ar can be phenyl, biphenyl, 1- or 2-naphthyl, 1- or 2-tetrahydrofuranyl, 2-, 3- or 4-pyridyl, 2- or 3-thienyl, 2- or 3-furyl, 2-, 4- or 5-thiazolyl, 2-, 4- or 5-oxazolyl, 1-pyrazolyl, 3-, 4- or 5-isoxazolyl, (C₃-C₆)-cycloalkyl, piperidinyl, pyrrolidinyl, oxopyridinyl, 2- or 3-pyrrolyl, 2- or 3-pyridazinyl, 2-, 4- or 5-pyrimidinyl, 2-pyrazinyl, 2-(1,3,5-triazinyl), 2-, 3- or 4-morpholinyl, 2- or 5-benzimidazolyl, 2-benzothiazolyl, 1,2,4-triazol-3-yl, 1,2,4-triazol-5-yl, tetrazol-5-yl, indol-3-yl, indol-5-yl or N-methyl-imidazol-2-, -4- or -5-yl and Ar may be substituted up to two times by F, Cl, Br, OH, CF₃, NO₂, CN, OCF₃, O—CH₂—O, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl, pyrrolidin-1-yl, morpholin-1-yl, piperidin-1-yl, piperazin-1-yl, 4-methyl-piperazin-1-yl, (CH₂)ₙ-phenyl, O—(CH₂)ₙ-phenyl, S—(CH₂)ₙ-phenyl, SO₂—(CH₂)ₙ-phenyl, where n=0-3;

Hal1, Hal2 independently of one another are F, Cl, Br.

For example, the invention includes intermediates of the following formula IV,

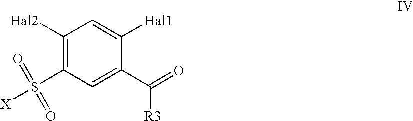

in which

X is (C₁-C₈)-alkyl, (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C₃-C₆)cycloalkyl, CONH₂, CONH(C₁-C₆)alkyl, CON[(C₁-C₆)alkyl]₂;

R3 independently of one another are NR6R7, (CH₂)-pyridyl, (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C₃-C₆)cycloalkyl, CONH₂, CONH(C₁-C₆)alkyl, CON[(C₁-C₆)alkyl]₂;

(C₁-C₈)-alkyl, pyrrolidine, piperidine, piperazine, piperazin-2-one, morpholine, tetrahydropyridine, tetrahydroquinoline, tetrahydroisoquinoline, where the rings may in each case be substituted by phenyl, (C₁-C₆)-alkyl-phenyl, —OH, (C₁-C₈)-alkyl, (C₁-C₆)-alkyl-OH, O-phenyl, S-phenyl, (CO)—(C₁-C₆)-alkyl, (CO)-phenyl, where the phenyl substituent is unsubstituted or substituted up to two times by F, Cl, Br, OH, CF₃, CN, OCF₃, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CON H(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl;

R6 and R7 independently of one another are H, (C₁-C₆)-alkyl, (C₁-C₆)-alkyl-OH, (C₁-C₆)-alkyl-NH2, (C₁-C₆)-alkyl-O—(C₁-C₆)-alkyl, O—(C₁-C₆)-alkyl, (C₃-C₆)-cycloalkyl, CO—(C₁-C₆)-alkyl, (C₁-C₆)-alkyl-NH—C(O)—(C₁-C₆)-alkyl, (C₁-C₆)-alkyl-NH—(C₁-C₆)-alkyl, (C₁-C₆)-alkyl-N—[(C₁-C₆)-alkyl]₂, (C₁-C₆)-alkyl-diphenyl, (C₁-C₆)-alkyl-O-phenyl, CHO, CO-phenyl,

(CH₂)n-Ar, where n=0-6 and Ar can be phenyl, biphenyl, 1- or 2-naphthyl, 1- or 2-tetrahydrofuranyl, 2-, 3- or 4-pyridyl, 2- or 3-thienyl, 2- or 3-furyl, 2-, 4- or 5-thiazolyl, 2-, 4- or 5-oxazolyl, 1-pyrazolyl, 3-, 4- or 5-isoxazolyl, (C₃-C₆)-cycloalkyl, piperidinyl, pyrrolidinyl, oxopyridinyl, 2- or 3-pyrrolyl, 2- or 3-pyridazinyl, 2-, 4- or 5-pyrimidinyl, 2-pyrazinyl, 2-(1,3,5-triazinyl), 2-, 3- or 4-morpholinyl, 2- or 5-benzimidazolyl, 2-benzothiazolyl, 1,2,4-triazol-3-yl, 1,2,4-triazol-5-yl, tetrazol-5-yl, indol-3-yl, indol-5-yl or N-methyl-imidazol-2-, -4- or -5-yl and Ar may be substituted up to two times by F, Cl, Br, OH, CF₃, N0₂, CN, OCF₃, O—CH₂—O, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl, pyrrolidin-1-yl, morpholin-1-yl, piperidin-1-yl, piperazin-1-yl, 4-methyl-piperazin-1-yl, (CH₂)ₙ-phenyl, O—(CH₂)ₙ-phenyl, S—(CH₂)ₙ-phenyl, SO₂—(CH₂)ₙ-phenyl, where n=0-3;

Hal1, Hal2 independently of one another are F, Cl, Br.

The examples given below serve to illustrate the invention, without restricting the scope of the invention.

1. Preparation of 4-chloro-2-fluoro-5-phenylmethanesulfonylbenzoic Acid

41 g (150 mmol) of 3-chlorosulfonyl-4-chloro-6-fluorobenzoic acid are added a little at a time to a solution of 18.8 g (150 mmol) of sodium sulfite in 230 ml of water. By addition of sodium hydroxide solution (33% in water), the pH is maintained at 7. The solution is stirred at 20-25° C. for another hour. The pH is adjusted to 2.0 using 2 N hydrochloric acid. 32 g (188 mmol) of benzyl bromide are added, and the mixture is stirred at 50° C. for 15 h. The suspension is cooled to 5° C. and the product is isolated by filtration and washed with a little water. The crude product is suspended in 200 ml of hot toluene. The suspension is cooled to 20° C. and the product is isolated by filtration and dried at 40° C. in a vacuum drying cabinet.

This gives 28 g of 4-chloro-2-fluoro-5-phenylmethanesulfonylbenzoic acid which are reacted further without further purification.

2. Preparation of 4-chloro-N,N-diethyl-2-fluoro-5-phenylmethanesulfonyl-benzamide

8.3 g (25.3 mmol) of the carboxylic acid from example 1 are suspended in 80 ml of toluene and 6 ml of thionyl chloride and, with stirring, heated under reflux for 1 hour. The mixture is then concentrated under reduced pressure using a rotary evaporator, the oily residue is dissolved in 100 ml of absolute dichloromethane and, at −10° C., 5.8 ml (2.2 equivalents) of diethylamine in 30 ml of dichloromethane are added dropwise. After the addition has ended, stirring is continued at 0° C. for 1 hour. The reaction mixture is then repeatedly washed, successively with saturated aqueous bicarbonate solution and water, and dried using sodium sulfate, and the solvent is removed under reduced pressure using a rotary evaporator. The crude product obtained in this manner is triturated with n-heptane, filtered off with suction and dried at 40° C. in a vacuum drying cabinet.

This gives 8.3 g of 4-chloro-N,N-diethyl-2-fluoro-5-phenylmethanesulfonyl-benzamide.

3. Preparation of 4-chloro-2-[(2-dimethylaminoethyl)ethylamino]-N,N-diethyl-5-phenylmethanesulfonylbenzamide

7.2 g (19 mmol) of 4-chloro-N,N-diethyl-2-fluoro-5-phenylmethanesulfonylbenzamide are dissolved in 60 ml of ethanol and, after addition of 4.5 ml (1.5 equivalents) of N-ethyl-N′,N′-dimethylethylenediamine, heated under reflux for 20 hours. The solvent is then removed under reduced pressure and the residue is taken up in 100 ml of dichloromethane and washed four times with in each case 40 ml of water. The organic phase is then dried over sodium sulfate and the solvent is removed under reduced pressure using a rotary evaporator.

This gives 9.1 g of a light-yellow oil which is directly converted into the end product of the reaction sequence.

4. Preparation of 2-[(2-dimethylaminoethyl)ethylamino]-N,N-diethyl-5-phenylmethanesulfonyl-4-(4-phenylpiperidin-1-yl)benzamide

8.9 g (18.6 mmol) of 4-chloro-2-[(2-dimethylaminoethyl)ethylamino]-N,N-diethyl-5-phenylmethanesulfonylbenzamide from experiment 3 are mixed with 14.5 g of 4-phenylpiperidin (4.8 equivalents), prepared by hydrogenation of commercially available 4-phenyl-1,2,3,6-tetrahydropyridine, and the mixture is stirred at 150° C. for 12 hours. 30 ml of ethyl acetate are then added. The solid is filtered off with suction and washed with 20 ml of ethyl acetate. The solvent is removed under reduced pressure using a rotary evaporator. For purification, the crude product is chromatographed on silica gel (40-63μ particle size, from Merck Darmstadt) as stationary phase, using ethyl acetate/methanol, mixing ratio 2:1. The crude product (9.9 g) is crystallized from diisopropyl ether.

This gives 8.6 g of 2-[(2-dimethylaminoethyl)ethylamino]-N,N-diethyl-5-phenylmethanesulfonyl-4-(4-phenylpiperidin-1-yl)benzamide, white crystals, m.p. 117-118° C., MS: C35H 48 N403 S (604.9); mass spectrum 605.3 (M+H⁺)

5. Preparation of 4-chloro-2-fluoro-5-methanesulfonylbenzoic Acid

5 g (18.3 mmol) of 3-chlorosulfonyl-4-chloro-6-fluorobenzoic acid are added to a solution of 2.77 g (22 mmol) of sodium sulfite in 55 ml of water. Over a period of 15 minutes, 5 g of a 33% strength aqueous sodium hydroxide solution are metered in. Following the addition of 4.3 g (45 mmol) of chloroacetic acid, the mixture is heated under reflux for 24 hours. The suspension is cooled and stirred at 0-5° C. for 1 hour. The product is filtered off with suction, washed with a little water and dried at 40° C. in a vacuum drying cabinet.

This gives 3.9 g of 4-chloro-2-fluoro-5-methanesulfonylbenzoic acid which are reacted further without further purification.

The further conversion of the resulting acid into compounds of the formula I is carried out analogously to examples 2 to 4.

We claim:

1. A process for preparing a sulfonylcarboxamide derivative of the formula I 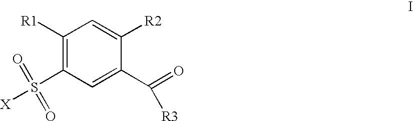
in which
X, R1, R2, R3 independently of one another are NR6R7, (CH₂)-pyridyl, (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C₃-C₆)cycloalkyl, CONH₂, CONH(C₁-C₆)alkyl or CON[(C₁-C₆)alkyl]₂;
(C₁-C₈)-alkyl, pyrrolidine, piperidine, piperazine, piperazin-2-one, morpholine, tetrahydropyridine, tetrahydroquinoline or tetrahydroisoquinoline, where the rings may in each case be substituted by phenyl, (C₁-C₆)-alkyl-phenyl, —OH, (C₁-C₈)-alkyl, (C₁-C₆)-alkyl-OH, O-phenyl, S-phenyl, (CO)—(C₁-C₆)-alkyl or (CO)-phenyl, where the phenyl substituent is unsubstituted or substituted up to two times by F, Cl, Br, OH, CF₃, CN, OCF₃, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CON H(C₃-C₆)cycloalkyl, NH₂, NH—CO—(C₁-C₆)-alkyl or NH—CO-phenyl;
R6 and R7 independently of one another are H, (C₁-C₆)-alkyl, (C₁-C₆)-alkyl-OH, (C₁-C₆)-alkyl-NH2, (C₁-C₆)-alkyl-O—(C₁-C₆)-alkyl, O—(C₁-C₆)-alkyl, (C₃-C₆)-cycloalkyl, CO—(C₁-C₆)-alkyl, (C₁-C₆)-alkyl-NH—C(O)—(C₁-C₆)-alkyl, (C₁-C₆)-alkyl-NH—(C₁-C₆)-alkyl, (C₁-C₆)-alkyl-N—[(C₁-C₆)-alkyl]₂, (C₁-C₆)-alkyl-diphenyl, (C₁-C₆)-alkyl-O-phenyl, CHO, CO-phenyl, or
(CH₂)ₙ—Ar, where n=0-6 and Ar can be phenyl, biphenyl, 1- or 2-naphthyl, 1- or 2-tetrahydrofuranyl, 2-, 3- or 4-pyridyl, 2- or 3-thienyl, 2- or 3-furyl, 2-, 4- or 5-thiazolyl, 2-, 4- or 5-oxazolyl, 1-pyrazolyl, 3-, 4- or 5-isoxazolyl, (C₃-C₆)-cycloalkyl, piperidinyl, pyrrolidinyl, oxopyridinyl, 2- or 3-pyrrolyl, 2- or 3-pyridazinyl, 2-, 4- or 5-pyrimidinyl, 2-pyrazinyl, 2-(1,3,5-triazinyl), 2-, 3- or 4-morpholinyl, 2- or 5-benzimidazolyl, 2-benzothiazolyl, 1,2,4-triazol-3-yl, 1,2,4-triazol-5-yl, tetrazol-5-yl, indol-3-yl, indol-5-yl or N-methyl-imidazol-2-, -4- or -5-yl and Ar may be substituted up to two times by F, Cl, Br, OH, CF₃, NO₂, CN, OCF₃, O—CH₂—O, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl, pyrrolidin-1-yl, morpholin-1-yl, piperidin-1-yl, piperazin-1-yl, 4-methyl-piperazin-1-yl, (CH₂)ₙ-phenyl, O—(CH₂)ₙ-phenyl, S—(CH₂)ₙ-phenyl or SO₂—(CH₂)ₙ-phenyl, where n=0-3;
which comprises preparing the compound of the formula I according to the reaction scheme below: 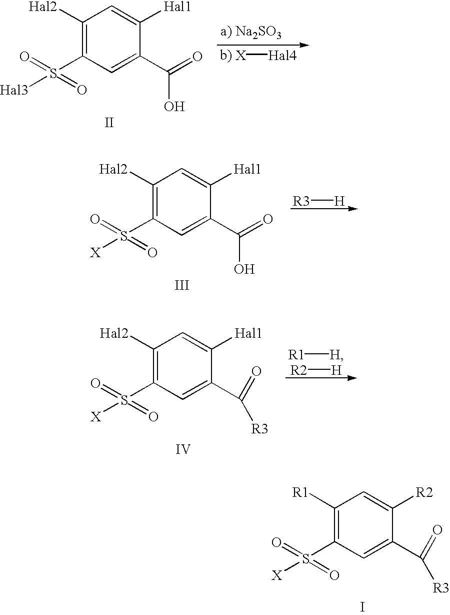
wherein
1) the compound of the formula II, in which Hall, Hal2 and Hal3 are each a halogen atom, is reduced using sodium sulfite and then, at a pH of from 1 to 3, reacted at from 0 to 80° C. in a suitable solvent with the compound X-Hal4, where X has the meaning given for formula I and Hal4 is a halogen atom, to give the compound of the formula III,
2) the compound of the formula III is reacted with the compound R3—H, in which R3 has the meaning given for formula 1, to give the compound of the formula IV, and
3) the compound of the formula IV is reacted with compounds R1—H and R2—H, where R1 and R2 have the meaning given for formula I, to give the compound of the formula I.

2. A process as claimed in claim 1, wherein Hall and Hal2 are, independently, each fluorine or chlorine.

3. A process as claimed in claim 1, wherein Hal3 is chlorine.

4. A process as claimed in claim 1, wherein the compound of the formula 11 is reduced using sodium sulfite, then reacted with the compound X-Hal4 at a pH of from 1.5 to 2.5.

5. A process as claimed in claim 1, wherein Hal4 is iodine, bromine or chlorine.

6. A process as claimed in claim 1, wherein Hal4 is bromine or chlorine.

7. A process as claimed in claim 1, wherein the suitable solvent comprises water, methanol, ethanol, propanol, butanol, dimethyl sulfoxide, dimethyl formamide, N-methylpyrrolidone or mixtures thereof.

8. A process as claimed in claim 1, wherein the compound of the formula II is reduced using sodium sulfite, then reacted with the compound X-Hal4 at a temperature of from 20 to 50° C.

9. A process for preparing a sulfonylcarboxamide derivative of the formula I 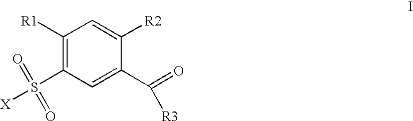
in which
X is CH₃;
R1, R2, R3 independently of one another are NR6R7, (CH₂)-pyridyl, (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C3-C₆)cycloalkyl, CONH₂, CONH(C₁-C₆)alkyl or CON[(C₁-C₆)alkyl]₂;
(C₁-C₈)-alkyl, pyrrolidine, piperidine, piperazine, piperazin-2-one, morpholine, tetrahydropyridine, tetrahydroquinoline or tetrahydroisoquinoline, where the rings may in each case be substituted by phenyl, (C₁-C₆)-alkyl-phenyl, —OH, (C₁-C₈)-alkyl, (C₁-C₆)-alkyl-OH, O-phenyl, S-phenyl, (CO)—(C₁-C₆)-alkyl or (CO)-phenyl, where the phenyl substituent is unsubstituted or substituted up to two times by F, Cl, Br, OH, CF₃, CN, OCF₃, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl or NH—CO-phenyl;
R6 and R7 independently of one another are H, (C₁-C₆)-alkyl, (C₁-C₆)-alkyl-OH, (C₁-C₆)-alkyl-NH2, (C₁-C₆)-alkyl-O—(C₁-C₆)-alkyl, O—(C₁-C₆)-alkyl, (C3-C₆)-cycloalkyl, CO—(C₁-C₆)-alkyl, (C₁-C₆)-alkyl-NH—C(O)—(C₁-C₆)-alkyl, (C₁-C₆)-alkyl-NH—(C₁-C₆)-alkyl, (C₁-C₆)-alkyl-N-[(C₁-C₆)-alkyl]₂, (C1-C6)-alkyl-diphenyl, (C₁-C₆)-alkyl-O-phenyl, CHO, CO-phenyl, or
(CH₂)ₙ—Ar, where n=0-6 and Ar can be phenyl, biphenyl, I- or 2-naphthyl, 1- or 2-tetrahydrofuranyl, 2-, 3- or 4-pyridyl, 2- or 3-thienyl, 2- or 3-furyl, 2-, 4- or 5-thiazolyl, 2-, 4- or 5-oxazolyl, 1-pyrazolyl, 3-, 4- or 5-isoxazolyl, (C₃-C₆)-cycloalkyl, piperidinyl, pyrrolidinyl, oxopyridinyl, 2- or 3-pyrrolyl, 2- or 3-pyridazinyl, 2-, 4- or 5-pyrimidinyl, 2-pyrazinyl, 2-(1,3,5-triazinyl), 2-, 3- or 4-morpholinyl, 2- or 5-benzimidazolyl, 2-benzothiazolyl, 1,2,4-triazol-3-yl, 1,2,4-triazol-5-yl, tetrazol-5-yl, indol-3-yl, indol-5-yl or N-methyl-imidazol-2-, -4- or -5-yl and Ar may be substituted up to two times by F, Cl, Br, OH, CF₃, NO₂, CN, OCF₃, O—CH₂—O, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl, pyrrolidin-1-yl, morpholin-1-yl, piperidin-1-yl, piperazin-1-yl, 4-methyl-piperazin-1-yl, (CH₂)n-phenyl, O—(CH₂)ₙ-phenyl, S—(CH₂)ₙ-phenyl or SO₂—(CH₂)ₙ-phenyl, where n=0-3;
which comprises preparing the compound of the formula I according to the reaction scheme below: 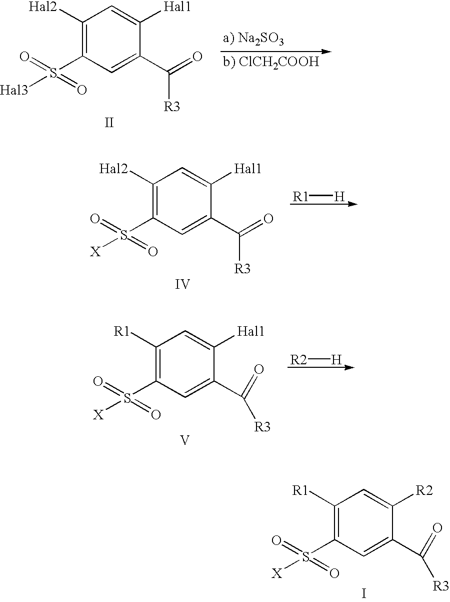
wherein
1) the compound of the formula II, in which R3 has the meaning given for formula I and Hal1, Hal2 and Hal3 are each a halogen atom, is reacted in the presence of sodium sulfite, at a pH of about 2, with chloroacetic acid, and the compound initially obtained of a general structure IVa, where X=CH₂CO₂H, is decarboxylated by heating and converted into the compound of the formula IV where X=CH₃,
2) the compound of the formula IV is reacted with a compound R1—H, where R1 has the meaning given for formula I, to give the compound of formula V, and
3) the compound of the formula V is reacted with a compound R2—H, in which R2 has the meaning given for formula i, to give the compound of the formula 1.

10. A process as claimed in claim 9, wherein Hal1, Hal2 and Hal3 are, independently, each fluorine or chlorine.

11. A process for preparing an intermediate of the formula IV, 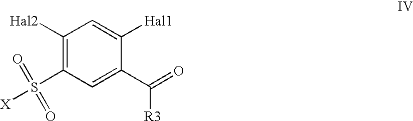
in which
X, R3 independently of one another are NR6R7, (CH₂)-pyridyl, (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C₃-C₆)cycloalkyl, CONH₂, CONH(C₁-C₆)alkyl or CON[(C₁-C₆)alkyl]₂;
(C₁-C₈)-alkyl, pyrrolidine, piperidine, piperazine, piperazin-2-one, morpholine, tetrahydropyridine, tetrahydroquinoline or tetrahydroisoquinoline, where the rings may in each case be substituted by phenyl, (C₁-C₆)-alkyl-phenyl, —OH, (C₁-C₈)-alkyl, (C₁-C₆)-alkyl-OH, O-phenyl, S-phenyl, (CO)—(C₁-C₆)-alkyl or (CO)-phenyl, where the phenyl substituent is unsubstituted or substituted up to two times by F, Cl, Br, OH, CF₃, CN, OCF₃, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl or NH—CO-phenyl;
R6 and R7 independently of one another are H, (C₁-C₆)-alkyl, (C₁-C₆)-alkyl-OH, (C₁-C₆)-alkyl-NH2, (C₁-C₆)-alkyl-O—(C₁-C₆)-alkyl, O—(C₁-C₆)-alkyl, (C₃-C₆)-cycloalkyl, CO—(C₁-C₆)-alkyl, (C₁-C₆)-alkyl-NH—C(O)—(C₁-C₆)-alkyl, (C₁-C₆)-alkyl-NH-(C₁-C₆)-alkyl, (C₁-C₆)-alkyl-N—[(C₁-C₆)-alkyl]₂, (C₁-C₆)-alkyl-diphenyl, (C₁-C₆)-alkyl-O-phenyl, CHO, CO-phenyl, or
(CH₂)ₙ—Ar, where n=0-6 and Ar can be phenyl, biphenyl, 1- or 2-naphthyl, 1- or 2-tetrahydrofuranyl, 2-, 3- or 4-pyridyl, 2- or 3-thienyl, 2- or 3-furyl, 2-, 4- or 5-thiazolyl, 2-, 4- or 5-oxazolyl, 1-pyrazolyl, 3-, 4- or 5-isoxazolyl, (C₃-C₆)-cycloalkyl, piperidinyl, pyrrolidinyl, oxopyridinyl, 2- or 3-pyrrolyl, 2- or 3-pyridazinyl, 2-, 4- or 5-pyrimidinyl, 2-pyrazinyl, 2-(1,3,5-triazinyl), 2-, 3- or 4-morpholinyl, 2- or 5-benzimidazolyl, 2-benzothiazolyl, 1,2,4-triazol-3-yl, 1,2,4-triazol-5-yl, tetrazol-5-yl, indol-3-yl, indol-5-yl or N-methyl-imidazol-2-, -4- or -5-yl and Ar may be substituted up to two times by F, Cl, Br, OH, CF₃, NO₂, CN, OCF₃, O—CH₂—O, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl, pyrrolidin-1-yl, morpholin-1-yl, piperidin-1-yl, piperazin-1-yl, 4-methyl-piperazin-1-yl, (CH₂)ₙ-phenyl, O—(CH₂)ₙ-phenyl, S—(CH₂)ₙ-phenyl or SO₂—(CH₂)ₙ-phenyl, where n=0-3;
Hal1, Hal2 independently of one another are F, Cl or Br; which comprises preparing the compound of the formula IV according to the reaction scheme below: 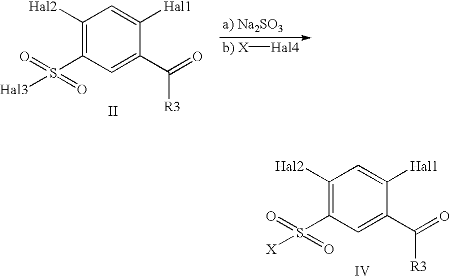
wherein
the compound of the formula II, in which R3 has the meaning given for formula IV and Hal1, Hal2 and Hal3 are each a halogen atom, is reacted in a suitable solvent and in the presence of sodium sulfite at a pH of about 2 with a compound X-Hal4, in which X has the meaning given for formula IV and Hal4 is a halogen atom, to give the compound of the formula IV.

12. A process as claimed in claim 11, wherein Hal1, Hal2 and Hal3 are, independently, fluorine or chlorine.

13. A process for preparing an intermediate of the formula IV, 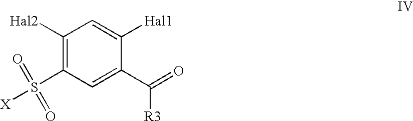
in which
X is CH₃;
R3 is NR6R7, (CH₂)-pyridyl, (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C₃-C₆)cycloalkyl, CONH₂, CONH(C₁-C₆)alkyl or CON[(C₁-C₆)alkyl]₂;
(C₁-C₈)-alkyl, pyrrolidine, piperidine, piperazine, piperazin-2-one, morpholine, tetrahydropyridine, tetrahydroquinoline or tetrahydroisoquinoline, where the rings may in each case be substituted by phenyl, (C₁-C₆)-alkyl-phenyl, —OH, (C₁-C₈)-alkyl, (C₁-C₆)-alkyl-OH, O-phenyl, S-phenyl, (CO)—(C₁-C₆)-alkyl or (CO)-phenyl, where the phenyl substituent is unsubstituted or substituted up to two times by F, Cl, Br, OH, CF₃, CN, OCF₃, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]2, CONH(C₃-C₆)cycloalkyl, NH₂, NH—CO—(C₁-C₆)-alkyl or NH—CO-phenyl;
R6 and R7 independently of one another are H, (C₁-C₆)-alkyl, (C₁-C₆)-alkyl-OH, (C₁-C₆)-alkyl-NH2, (C₁-C₆)-alkyl-O—(C1-C6)-alkyl, O—(C₁-C₆)-alkyl, (C₃-C₆)-cycloalkyl, CO—(C₁-C₆)-alkyl, (C₁-C₆)-alkyl-NH—C(O)—(C₁-C₆)-alkyl, (C₁-C₆)-alkyl-NH—(C₁-C₆)-alkyl, (C₁-C₆)-alkyl-N—[(C1-C6)-alkyl]2, (C₁-C₆)-alkyl-diphenyl, (C₁-C₆)-alkyl-O-phenyl, CHO, CO-phenyl, or
(CH₂)ₙ—Ar, where n=0-6 and Ar can be phenyl, biphenyl, 1- or 2-naphthyl, 1- or 2-tetrahydrofuranyl, 2-, 3- or 4-pyridyl, 2- or 3-thienyl, 2- or 3-furyl, 2-, 4- or 5-thiazolyl, 2-, 4- or 5-oxazolyl, 1-pyrazolyl, 3-, 4- or 5-isoxazolyl, (C₃-C₆)-cycloalkyl, piperidinyl, pyrrolidinyl, oxopyridinyl, 2- or 3-pyrrolyl, 2- or 3-pyridazinyl, 2-, 4- or 5-pyrimidinyl, 2-pyrazinyl, 2-(1,3,5-triazinyl), 2-, 3- or 4-morpholinyl, 2- or 5-benzimidazolyl, 2-benzothiazolyl, 1,2,4-triazol-3-yl, 1,2,4-triazol-5-yl, tetrazol-5-yl, indol-3-yl, indol-5-yl or N-methyl-imidazol-2-, -4- or -5-yl and Ar may be substituted up to two times by F, Cl, Br, OH, CF₃, N0₂, CN, OCF₃, O—CH₂—O, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl, pyrrolidin-1-yl, morpholin-1-yl, piperidin-1-yl, piperazin-1-yl, 4-methyl-piperazin-1-yl, (CH₂)ₙ-phenyl, O—(CH₂)ₙ-phenyl, S—(CH₂)ₙ-phenyl or SO₂—(CH₂)ₙ-phenyl, where n=0-3;
Hal1, Hal2 independently of one another are F, Cl or Br;
which comprises preparing the compound of the formula IV according to the reaction scheme below: 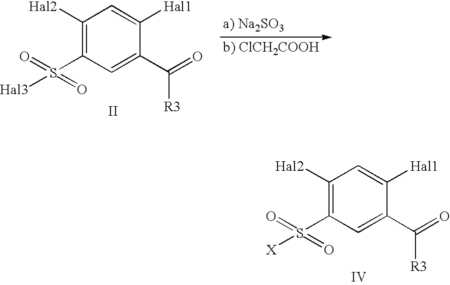
wherein
the compound of the formula II, in which R3 has the meaning given for formula IV and Hal1, Hal2 and Hal3 are each a halogen atom, is reacted in a suitable solvent and in the presence of sodium sulfite at a pH of about 2 with chloroacetic acid to give the compound of the formula IV.

14. A process as claimed in claim 13, wherein Hal1, Hal2 and Hal3 are, independently, fluorine or chlorine.

15. An intermediate of the formula IV, 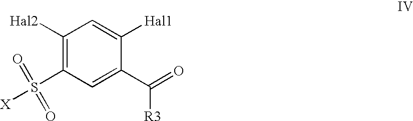
in which
X, R3 independently of one another are NR6R7, (CH₂)-pyridyl, (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C₃-C₆)cycloalkyl, CONH₂, CONH(C₁-C₆)alkyl or CON[(C₁-C₆)alkyl]₂;
(C₁-C₈)-alkyl, pyrrolidine, piperidine, piperazine, piperazin-2-one, morpholine, tetrahydropyridine, tetrahydroquinoline or tetrahydroisoquinoline, where the rings may in each case be substituted by phenyl, (C₁-C₆)-alkyl-phenyl, —OH, (C₁-C₈)-alkyl, (C₁-C₆)-alkyl-OH, O-phenyl, S-phenyl, (CO)—(C₁-C₆)-alkyl or (CO)-phenyl, where the phenyl substituent is unsubstituted or substituted up to two times by F, Cl, Br, OH, CF₃, CN, OCF₃, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]2, CONH(C₃-C₆)cycloalkyl, NH₂, NH—CO—(C₁-C₆)-alkyl or NH—CO-phenyl;
R6 and R7 independently of one another are H, (C₁-C₆)-alkyl, (C₁-C₆)-alkyl-OH, (C₁-C₆)-alkyl-NH2, (C₁-C₆)-alkyl-O—(C₁-C₆)-alkyl, O—(C₁-C₆)-alkyl, (C₃-C₆)-cycloalkyl, CO—(C₁-C₆)-alkyl, (C₁-C₆)-alkyl-NH—C(O)—(C₁-C₆)-alkyl, (C₁-C₆)-alkyl-NH—(C₁-C₆)-alkyl, (C₁-C₆)-alkyl-N—[(C₁-C₆)-alkyl]₂, (C₁-C₆)-alkyl-diphenyl, (C₁-C₆)-alkyl-O-phenyl, CHO, CO-phenyl, or (CH₂)ₙ—Ar, where n=0-6 and Ar can be phenyl, biphenyl, 1- or 2-naphthyl, 1- or 2-tetrahydrofuranyl, 2-, 3- or 4-pyridyl, 2- or 3-thienyl, 2- or 3-furyl, 2-, 4- or 5-thiazolyl, 2-, 4- or 5-oxazolyl, 1-pyrazolyl, 3-, 4- or 5-isoxazolyl, (C₃-C₆)-cycloalkyl, piperidinyl, pyrrolidinyl, oxopyridinyl, 2- or 3-pyrrolyl, 2- or 3-pyridazinyl, 2-, 4- or 5-pyrimidinyl, 2-pyrazinyl, 2-(1,3,5-triazinyl), 2-, 3- or 4-morpholinyl, 2- or 5-benzimidazolyl, 2-benzothiazolyl, 1,2,4-triazol-3-yl, 1,2,4-triazol-5-yl, tetrazol-5-yl, indol-3-yl, indol-5-yl or N-methyl-imidazol-2-, -4- or -5-yl and Ar may be substituted up to two times by F, Cl, Br, OH, CF₃, N0₂, CN, OCF₃, O—CH₂—O, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl, pyrrolidin-1-yl, morpholin-1-yl, piperidin-1-yl, piperazin-1-yl, 4-methyl-piperazin-1-yl, (CH₂)ₙ-phenyl, O—(CH₂)ₙ-phenyl, S—(CH₂)ₙ-phenyl or SO₂—(CH₂)ₙ-phenyl, where n=0-3;
Hal1, Hal2 independently of one another are F, Cl or Br.

16. An intermediate of the formula IV as claimed in claim 15, wherein the radicals have the following meaning
X is (C₁-C₈)-alkyl or (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C₃-C₆)cycloalkyl, CONH₂, CONH(C₁-C₆)alkyl or CON[(C₁-C₆)alkyl]₂;
R3 is NR6R7, (CH₂)-pyridyl or (CH₂)ₙ-phenyl, where n=0-6 and the phenyl radical may be substituted up to two times by F, Cl, Br, CF₃, NH₂, CN, OCF₃, O—(C₁-C₆)-alkyl, S—(C₁-C₆)-alkyl, (C₁-C₆)-alkyl, (C₃-C₆)-cycloalkyl, COO(C₁-C₆)-alkyl, COO(C₃-C₆)cycloalkyl, CONH₂, CONH(C₁-C₆)alkyl or CON[(C₁-C₆)alkyl]₂;
(C₁-C₈)-alkyl, pyrrolidine, piperidine, piperazine, piperazin-2-one, morpholine, tetrahydropyridine, tetrahydroquinoline or tetrahydroisoquinoline, where the rings may in each case be substituted by phenyl, (C₁-C₆)-alkyl-phenyl, —OH, (C₁-C₈)-alkyl, (C₁-C₆)-alkyl-OH, O-phenyl, S-phenyl, (CO)—(C₁-C₆)-alkyl or (CO)-phenyl, where the phenyl substituent is unsubstituted or substituted up to two times by F, Cl, Br, OH, CF₃, CN, OCF₃, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl or NH—CO-phenyl;
R6 and R7 independently of one another are H, (C₁-C₆)-alkyl, (C₁-C₆)-alkyl-OH, (C₁-C₆)-alkyl-NH2, (C₁-C₆)-alkyl-O—(C₁-C₆)-alkyl, O—(C₁-C₆)-alkyl, (C₃-C₆)-cycloalkyl, CO—(C₁-C₆)-alkyl, (C₁-C₆)-alkyl-NH—C(O)—(C₁-C₆)-alkyl, (C₁-C₆)-alkyl-NH—(C₁-C₆)-alkyl, (C₁-C₆)-alkyl-N—[(C₁-C₆)-alkyl]₂, (C₁-C₆)-alkyl-diphenyl, (C₁-C₆)-alkyl-O-phenyl, CHO, CO-phenyl, or
(CH₂)ₙ—Ar, where n=0-6 and Ar can be phenyl, biphenyl, 1- or 2-naphthyl, 1- or 2-tetrahydrofuranyl, 2-, 3- or 4-pyridyl, 2- or 3-thienyl, 2- or 3-furyl, 2-, 4- or 5-thiazolyl, 2-, 4- or 5-oxazolyl, 1-pyrazolyl, 3-, 4- or 5-isoxazolyl, (C₃-C₆)-cycloalkyl, piperidinyl, pyrrolidinyl, oxopyridinyl, 2- or 3-pyrrolyl, 2- or 3-pyridazinyl, 2-, 4- or 5-pyrimidinyl, 2-pyrazinyl, 2-(1,3,5-triazinyl), 2-, 3- or 4-morpholinyl, 2- or 5-benzimidazolyl, 2-benzothiazolyl, 1,2,4-triazol-3-yl, 1,2,4-triazol-5-yl, tetrazol-5-yl, indol-3-yl, indol-5-yl or N-methyl-imidazol-2-, -4- or -5-yl and Ar may be substituted up to two times by F, Cl, Br, OH, CF₃, NO₂, CN, OCF₃, O—CH₂—O, O—(C₁-C₆)-alkyl, S—(C₁-C₆)-alkyl, SO—(C₁-C₆)-alkyl, SO₂—(C₁-C₆)-alkyl, (C₁-C₆)-alkyl, (C₃-C₆)-cycloalkyl, COOH, COO(C₁-C₆)alkyl, COO(C₃-C₆)cycloalkyl, CONH₂, CONH(C₁-C₆)alkyl, CON[(C₁-C₆)alkyl]₂, CONH(C₃-C₆)cycloalkyl, NH₂, NH—CO—(C₁-C₆)-alkyl, NH—CO-phenyl, pyrrolidin-1-yl, morpholin-1-yl, piperidin-1-yl, piperazin-1-yl, 4-methyl-piperazin-1-yl, (CH₂)ₙ-phenyl, O—(CH₂)ₙ-phenyl, S—(CH₂)ₙ-phenyl or SO₂—(CH₂)ₙ-phenyl, where n=0-3;
Hal1, Hal2 independently of one another are F, Cl or Br.
